(19) United States
(12) Patent Application Publication
(10) Pub. No.: US 2020/0171263 A1
(43) Pub. Date: Jun. 4, 2020

(54) BREATH WARMER AND SENSOR PLATFORM

(71) Applicant: SOLATIDO, INC., Palo Alto, CA (US)

(72) Inventor: Thomas Mallory SHERLOCK, Los Altos, CA (US)

(21) Appl. No.: 16/703,068

(22) Filed: Dec. 4, 2019

Related U.S. Application Data
(60) Provisional application No. 62/775,325, filed on Dec. 4, 2018.

(51) Int. Cl. A61M 16/10; A62B 18/10; A62B 18/02

(52) U.S. Cl. CPC A61M 16/1045; A61M 16/0003; A62B 18/02; A62B 18/10

(57) ABSTRACT

A breathing device that warms inhaled air by absorbing the heat of exhaled air into one side of a metallic membrane that transfers that heat to incoming air on the other side of the membrane. Flow of inhaled and exhaled air is separated by the membrane and is controlled by check valves. The volume of exhaled air that must be inhaled again before getting any fresh air is thereby minimized. The primary purpose is to warm inhaled air, but sensors within it can facilitate gas analysis on exhaled air to diagnose and predict various diseases. An electric heater, vibrational de-icer, and humidifier are additional options.

### CROSS-REFERENCE TO RELATED APPLICATIONS

This application claims benefit of U.S. provisional patent application Ser. No. 62/775,325, the contents are set forth herein in their entirety.

### BACKGROUND

### Breath Warmers:

The breathing of cold air is uncomfortable and even dangerous for some, depending upon the severity of the temperature, and a person's physical health. In some situations one's survival can depend on the ability to retain body heat. There is also a danger of damage to the lung tissue in some instances. There are also specific cases in which excessive breathing of cold air can trigger heart attack, or asthma. Aside from the discomfort, inhalation of cold air can make one more vulnerable to Infection. Scarves and more sophisticated headgear have been used to lessen the effect of the cold air by retaining a bit of the heat of exhaled air, to transfer it to the incoming air, but most of these allow the incoming air to retrace the same path as the air that was just exhaled. Therefore, any residual exhaled air that is trapped in the matrix of a scarf or simple breath warmer will have to be inhaled before any fresh, oxygen-rich air is consumed.

### Exhaled Gas Sensors:

Until recently, sensors for analyzing exhaled gasses have been available primarily in the clinical setting, since the equipment has been large, complicated, expensive, and fragile. Recently, various robust sensors have become available that can be embedded into portable equipment. This is useful for professionals such as paramedics, but there has been little reason for individuals to carry such equipment for self-analysis unless they have a particularly dangerous condition that warrants such care.

This device can take several forms, including a face mask that covers the mouth and nose, or a remote housing that is connected via hoses or other conduits to mouth or face interfaces. It's primary purpose is to warm the inhaled air, but with the addition of electronics, a secondary use, in some configurations, will be to do gas analysis on exhaled air to monitor various bodily states and diagnose and predict various diseases. A sniffer circuit, auxiliary heater, vibrational de-icer, and humidifier can be added depending upon the severity of the climate and the user's activity level. Aside from the comfort of warmed incoming air, there are actual dangers posed by cold air inhalation during exertion. Asthma and arterial thrombosis are two of the particular problems aggravated by breathing cold air, often during physical exertion such as snow shoveling.

Sensors for exhaled gasses can provide data for diagnosis of various other maladies. Accurate detection of specific VOCs (volatile organic compounds) in exhaled breath, known as biomarkers, can provide essential information for the diagnosis of specific diseases. This sensor data can be communicated to a Smartphone or other device over a WiFi or Bluetooth connection, for analysis and relay of results and warnings.

### BRIEF DESCRIPTION OF THE DRAWINGS

FIG. 1 shows one embodiment of a basic assembly;

FIG. 2 shows a heat exchanger of the basic assembly;

FIG. 3 shows more detail of the basic assembly;

FIGS. 4 through 6 show a dual heat exchanger embodiment;

FIGS. 7 and 8 show a dual system with humidity foam blocks;

FIGS. 9 and 10 show a basic embodiment using flat metallic sheets for the heat exchanger instead of fan folded sheet metal;

FIGS. 11 and 12 show flat sheets and gaskets used to build up a heat exchanger in a dual heat exchanger system; and

FIGS. 13-15 show further detail of a dual system with two stacks of flat sheets.

### DETAILED DESCRIPTION

A face-worn, or otherwise accessible breathing device provides a portable means of warming inhaled air, using the waste heat of the user's exhaled breath, while also providing a platform for the mounting and interrogation of inhaled and exhaled breath sensors that can diagnose and quantify various diseases and dangers. Sensors can be included to gather data regarding ambient conditions, allowing for calculation of whether heat or vibration needs to be deployed for de-icing or comfort.

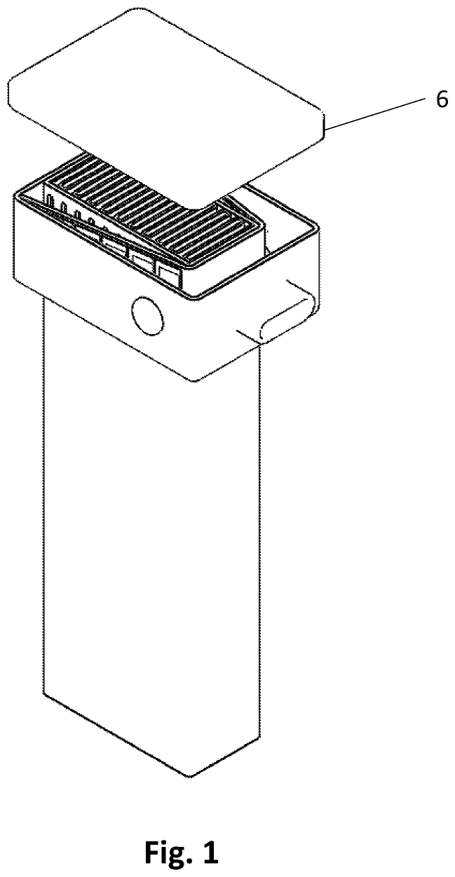

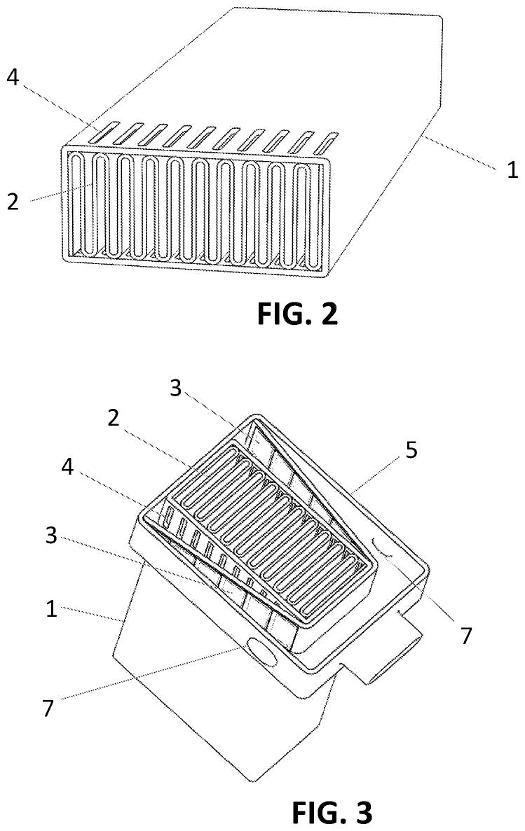

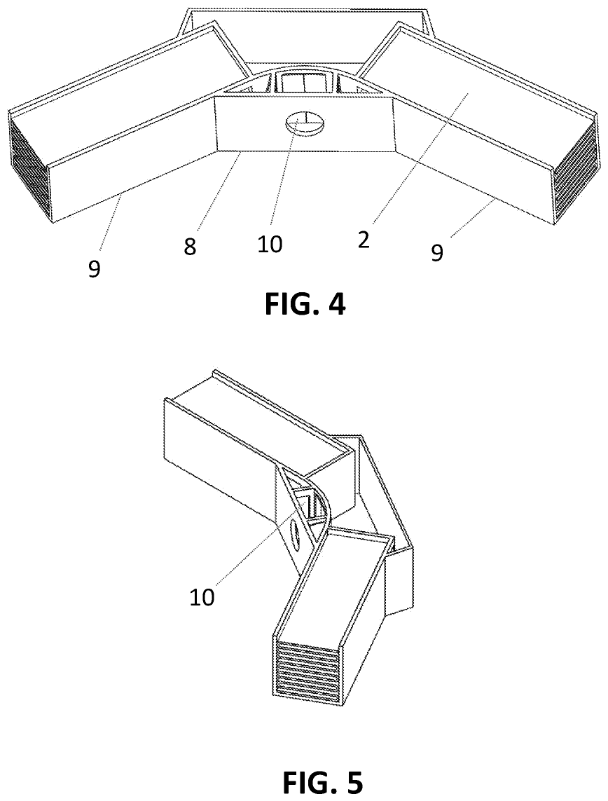

FIGS. 1-3 show a basic embodiment of the device that warms inhaled air as it passes through a tube 1 by absorbing the heat of exhaled air into one side of a thermally conductive, fan folded membrane 2 that subsequently gives up that absorbed heat to incoming air on the other side of the membrane. Flow of inhaled and exhaled air is separated by the membrane 2 and controlled by at least two check valves 3 that are adjacent to slots 4 in the tube 1 and close to the user's face, to minimize the volume of exhaled air that must be inhaled again before getting any fresh air. One end of the tube 1 and membrane 2 is encased in a housing 5 sealed by a cover 6, that forces air from either side of the membrane to pass through the slots 4 and the check valves 3, thereby separating the exhaled air from the inhaled air. Sensors 7, used to analyze inhaled and exhaled gasses are mounted on the sides of the housing.

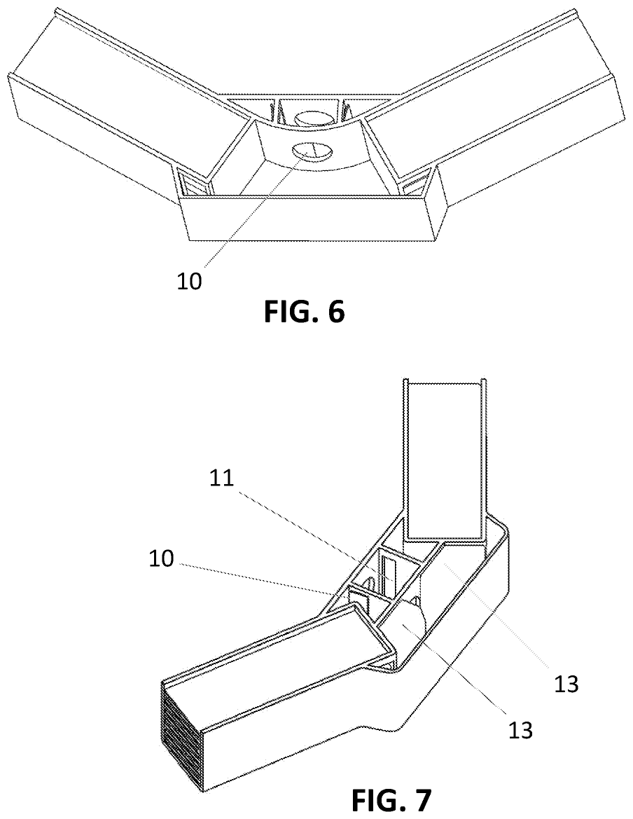

A more wearable embodiment is shown in FIGS. 4, 5 and 6. A dual tube housing 8 is shown with a tube 9 extending from either side of the housing 8. A membrane 2 is in each of the tubes 9. Check valves 10 are places over slots 11 in the housing 8 to manage the directional flow of incoming air and exhaled breath.

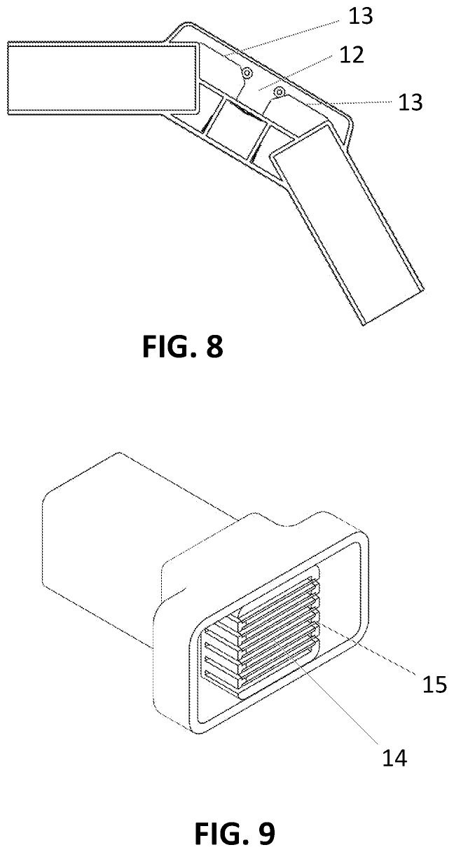

FIGS. 7 and 8 show how humidity may be added to incoming air, when necessary, by passing air through a humidifying chamber 12 that contains one or more moistened absorbent blocks 13 that hold an adequate amount of water for the expected time of use.

In some instances, temperature gradients permitting, humidity can be recirculated from exhaled gasses, by collecting moisture on a liquid permeable portion of the membrane, to be delivered on the other side of the membrane to the incoming air. For extended use in cold weather, exhaled humidity can be condensed onto non-freezing fins that drain that moisture into a humidification container to be used to humidify the incoming air.

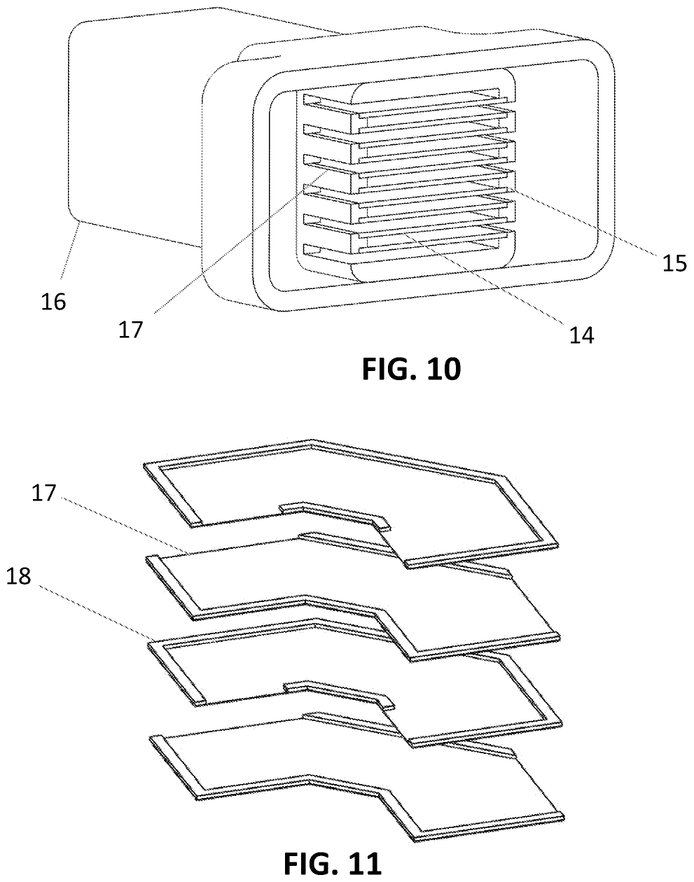

FIGS. 9 and 10 show an alternate construction of the basic, single tube embodiment with the cover, and check valves omitted for clarity. Instead of fan folding the sheet metal that forms the membrane, discreet sheets 14 are inserted into grooves 15 that are formed into the interior sides of an extruded tube 16. Slots 17 are machined or punched in the sides of one end of the extruded tube 16 in a manner that mimics the fan folded approach by alternating the slots such that incoming air goes one direction when exiting the extruded tube, and outgoing breath enters from the other direction. A cover containing check valves and a mouthpiece is added to this assembly to complete it.

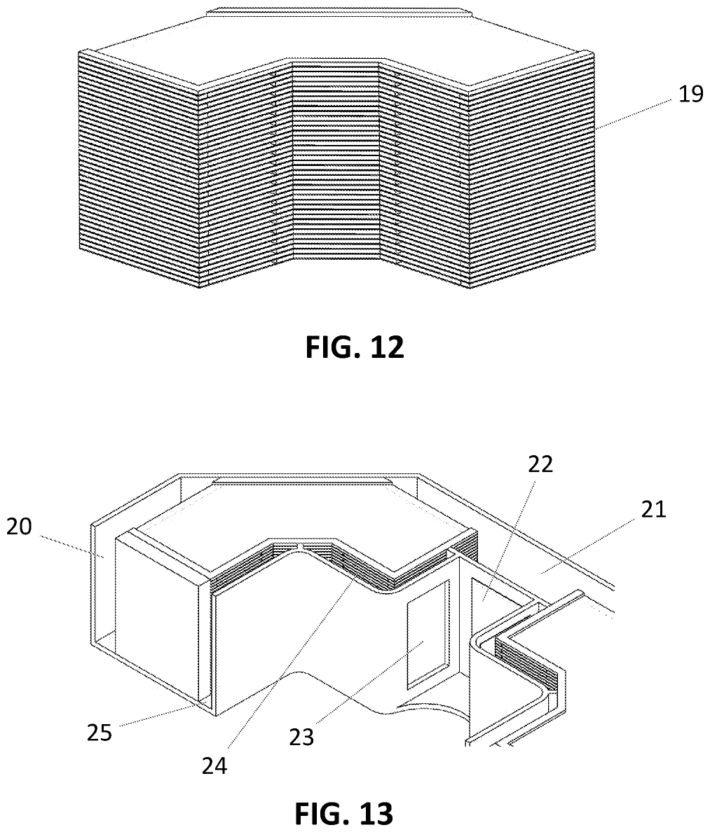

FIG. 11 shows another embodiment of the device using a number of flat conductive sheets 17 that contain gasket material 18 to seal against the next sheet in a manner that alternates the side at which air or exhaled breath will enter or exit a stack 19 once the parts are tightly stacked together. This gasket can be applied using adhesive, or may be formed into each sheet itself to contact the sheet above it, thereby sealing each level, and preventing the gasses from shorting into the incorrect gas circuit.

FIG. 12 shows a number of these sheets 17 tightly assembled into a stack 19 for assembly into the next higher assembly.

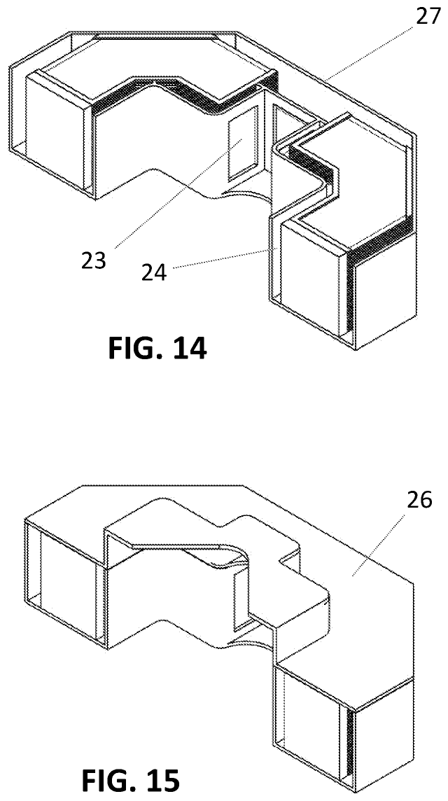

FIG. 13 shows one side of the next higher assembly, without its cover, in which a housing envelopes at least one stack 19 of sheets 17 and provides for management of gasses through and around the stacks of sheets. Incoming air enters through the entry plenum 20 and is directed into every second interstitial space within the stack of sheets, exiting into a common incoming air chamber 21. From here, the air passes through an incoming air check valve 22 and out of the assembly into the user's mouth and nose. Exhaled breath passes from the user into the assembly through the outgoing breath check valves 23, then into the outgoing breath plenum 24, and then through the stack, to transfer the warmth of the outgoing breath into the sheets, and then from the stack into the exit plenum 25, and out to the ambient atmosphere.

FIG. 14 shows a complete assembly of the previous figure, except for a cover.

FIG. 15 shows the assembly with a cover 26 to seal the airflow of the various passageways, and provide for whatever soft material might be added to comfortably conform to the face of the user.

Exhaled gasses exit away from the intake, to avoid freezing the outbound humidity onto the material that is being cooled by the incoming ambient air. Some longitudinal conduction can keep the heat exchanger warm enough to avoid freezing at the output as long as the incoming air is warmed a bit first. This can be further ensured by coating a portion of the outgoing air side of the exchanger with a non-stick material that conducts heat poorly, such as polyethylene or PTFE (TEFLON, CHEMOURS, Wilmington, Del.), so that edges that are proximate to cold air may be slightly insulated and hydrophobic, and more easily de-iced if humidity builds up on the outer edges of the exhaled breath path.

Regardless of whether the conductive membrane is made of a fan folded sheet or discreet sheets that are stacked, copper and aluminum are the primary candidates for the conductive membrane. For any given configuration, their value as a heat exchanger is optimized for weight, cost, effectiveness, durability, and ease of cleaning.

Foamed NEOPRENE (CHEMOURS, Wilmington, Del.) covers, of the variety that is found in wetsuits, may be used to insulate the assembly if desired. A forward portion of the housing may be made from a thin diaphragm 27 that allows speech to be heard through it, when not covered by an insulating flap.

In these embodiments, parts other than the heat conductive membrane may be fabricated from one or more thermoplastic polymers, such as Polycarbonate, Acrylonitrile butadiene styrene (ABS), or Polyethylene, in order to keep undesirable conduction to a minimum, thereby conserving the heat for warming the incoming air.

In these embodiments, the effectiveness of the warming properties at any given ambient temperature and activity level is a function of the length of the air path and number of layers of membrane. For much colder climates, a longer air path with more layers is desirable. This allows for a longer dwell time for exhaled gasses within the interstitial spaces to transfer their heat to the membrane, and therefore the incoming air, before being discarded.

In embodiments, an exterior flexible membrane allows voice vibrations to be transmitted out of the face mask, such that the wearer can verbally communicate with others.

Filters may be installed in various stages of the device to ensure proper inhaled air quality.

What is claimed is:

1. A breath warming apparatus comprising:
a housing;
at least one heat conductive membrane dividing the inner space of the housing into at least two spaces;
a plurality of check valves disposed on either side of the membrane such that air can only travel in one direction across either side of the membrane; and
a means of connecting the housing to a person's airway.

2. The apparatus of claim 1, further comprising a face mask that encircles the mouth and nose of a person.

3. The apparatus of claim 2, further comprising an exterior flexible membrane that allows voice vibrations to be transmitted out of the face mask, such that the wearer can verbally communicate with others.

4. The apparatus of claim 3, further comprising an exterior flap allowing for selective insulation of the flexible membrane.

5. The apparatus of claim 1, further comprising a breathing tube that can be held in the person's mouth

6. The apparatus of claim 3, the breathing tube comprising a divided breathing tube that separates incoming air from outgoing air.

7. The apparatus of claim 1, further comprising a membrane that is made of a metal.

8. The apparatus of claim 1, further comprising a membrane that is fan folded.

9. The apparatus of claim 8, further comprising openings in the housing that are disposed opposite to each other at one end of the fan folded membrane such that when the cut edge of the fan form is blocked, the air traveling across one side of the folds will exit one of the openings without mixing with the air that is entering the other opening.

10. The apparatus of claim 9, the membrane comprising a membrane that is built up of stacked layers of sheets that are joined by interstitial gaskets that define the incoming air passages and the outgoing air passages.

11. The apparatus of claim 1, further comprising a membrane that is built up of stacked layers of sheets.

12. The apparatus of claim 1, further comprising at least one sensor.

13. The apparatus of claim 12, wherein the sensor is capable of detecting at least one of the substances selected from the following list:
CO₂;
ketones;
volatile organic compounds (VOC);
hydrogen sulfide;
acetone;
toluene;
ammonia;
nitrogen monoxide; and
pentane.

14. The apparatus of claim 1, further comprising a heating element.

15. The apparatus of claim 1, further comprising a flexible insulating cover.

16. The apparatus of claim 1, further comprising a battery.

17. The apparatus of claim 1, further comprising a heat conductive membrane with a non-stick surface finish that resists ice build-up.

18. The apparatus of claim 1, further comprising a membrane with a non-stick surface finish made by depositing hydrophobic plastic onto at least a portion of a conductive metal sheet.
